(19) United States
(12) Patent Application Publication
(10) Pub. No.: US 2024/0301397 A1
(43) Pub. Date: Sep. 12, 2024

(54) SYSTEMS AND METHODS FOR CAPTURE OF CIRCULATING FREE DNA

(71) Applicant: The Johns Hopkins University, Baltimore, MD (US)

(72) Inventor: Bradley Downs, Baltimore, MD (US)

(21) Appl. No.: 18/291,122

(22) PCT Filed: Jul. 22, 2022
(86) PCT No.: PCT/US2022/074052

§ 371 Date: Jan. 22, 2024

Related U.S. Application Data
(60) Provisional application No. 63/225,034, filed on Jul. 23, 2021.

(51) Int. Cl. C12N 15/10; C12N 9/22; C12N 15/11

(52) U.S. Cl. CPC C12N 15/1013; C12N 9/22; C12N 15/11; C12N 2310/20

(57) ABSTRACT

Through a combination of apoptosis, necrosis, and secretion, tumor DNA is released into the bloodstream and becomes part of the cell-free DNA (cfDNA). The present disclosure provides methods, systems, devices, and kits for isolating circulating free DNA (cfDNA) from a biological sample (e.g., plasma) using RNA-guided DNA binding proteins (e.g. Gas proteins.). Particularly, the disclosure provide methods, systems, devices, and kits for Cas9 mediated capture of cfDNA from flowing plasma.

### CROSS-REFERENCE TO RELATED APPLICATIONS

This application claims the benefit of U.S. Provisional Application No. 63/225,034, filed Jul. 23, 2021, the content of which is herein incorporated by reference in its entirety.

### STATEMENT REGARDING FEDERALLY-SPONSORED RESEARCH OR DEVELOPMENT

This invention was made with government support under grant number W81XWH1810482 awarded by the Department of Defense. The government has certain rights in the invention.

### FIELD

The present disclosure provides methods, systems, and kits for isolating circulating free DNA (cfDNA) from a biological sample (e.g., plasma) using RNA-guided DNA binding proteins (e.g., Cas proteins).

### SEQUENCE LISTING STATEMENT

The contents of the electronic sequence listing titled JHU-39649-601.xml (Size: 26,172 bytes; and Date of Creation: Jul. 22, 2022) is herein incorporated by reference in its entirety.

### BACKGROUND

Through a combination of apoptosis, necrosis, and secretion, tumor DNA is released into the bloodstream and becomes part of the cell-free DNA (cfDNA). Cancer patients have a wide range of levels of altered tumor-specific DNA, from undetectable levels to more than 80% of the total circulating cfDNA. The levels of circulating tumor DNA (cfDNA) vary with tumor stage and size among other factors. Thus, analysis of cfDNA provides an opportunity to noninvasively detect early cancer, track changes in tumor burden and to monitor response to treatment. However, a limiting factor for early detection is the small number of cfDNA in the blood and as the concentration of cfDNA decreases, the volume of blood required to detect the cfDNA increases to prohibitive levels.

### SUMMARY

Disclosed herein are methods, systems, devices, and kits for capturing circulating free DNA from a biological sample of a subject.

In some embodiments, the methods comprise forming a capture complex comprising an RNA-guided DNA binding protein, or a functional fragment thereof, bound to a guide RNA configured to at least partially hybridize to a circulating free DNA of interest; incubating the capture complex with the biological sample to form a bound complex comprising the capture complex bound to the circulating free DNA of interest; and removing remaining biological sample from the bound complex. In some embodiments, the incubating comprises flowing the biological sample over immobilized capture complexes.

In some embodiments, the methods further comprise: purifying the circulating free DNA of interest; amplifying the circulating free DNA of interest; sequencing the circulating free DNA of interest; or a combination thereof.

In some embodiments, the methods further comprise analyzing at least a portion of the biological sample for the presence or absence of at least one biomarker.

In some embodiments, the methods further comprise returning at least a portion of the biological sample to the subject.

In some embodiments, the systems comprise an RNA-guided DNA binding protein, or a functional fragment thereof; and a guide RNA configured to at least partially hybridize to a circulating free DNA of interest. The systems may further comprise one or more of: a flow cell or fluidic chamber, at least one magnet, a solid surface, and a biological sample.

In some embodiments, the biological sample comprises blood or blood components. In some embodiments, the blood component comprises plasma.

In some embodiments, the circulating free DNA is of microbial or viral origin. In some embodiments, the circulating free DNA is circulating tumor DNA.

In some embodiments, the capture complex is linked to a solid surface. In some embodiments, the solid surface is a particle. In some embodiments, the particle is magnetic. In some embodiments, the capture complex is linked to the solid surface by a biotin streptavidin linker. In some embodiments, the RNA-guided DNA binding protein further comprises a biotin tag. In some embodiments, the RNA-guided DNA binding protein comprises a FLAG tag.

In some embodiments, the RNA-guided DNA binding protein is a CRISPR-associated (Cas) protein. In some embodiments, the RNA-guided DNA binding protein is Cas9. In some embodiments, the Cas9 is catalytically inactivated.

Also provided herein are devices comprising a system as disclosed herein. In some embodiments, the devices comprise two or more systems. In certain embodiments, each system of the device comprises a gRNA configured to hybridize to a different circulating fee DNA of interest.

Other aspects and embodiments of the disclosure will be apparent in light of the following detailed description and accompanying figures.

### BRIEF DESCRIPTION OF THE DRAWINGS

FIGS. 1A-IC show visualization of the cfDNA capture technology. FIG. 1A is a schematic of how the cfDNA is captured with flow in the p-slide luer, shown in FIG. 1B. FIG. 1C is a schematic of pulling down dCas9 bound to metal streptavidin beads in a 0.5 ml PCR tube.

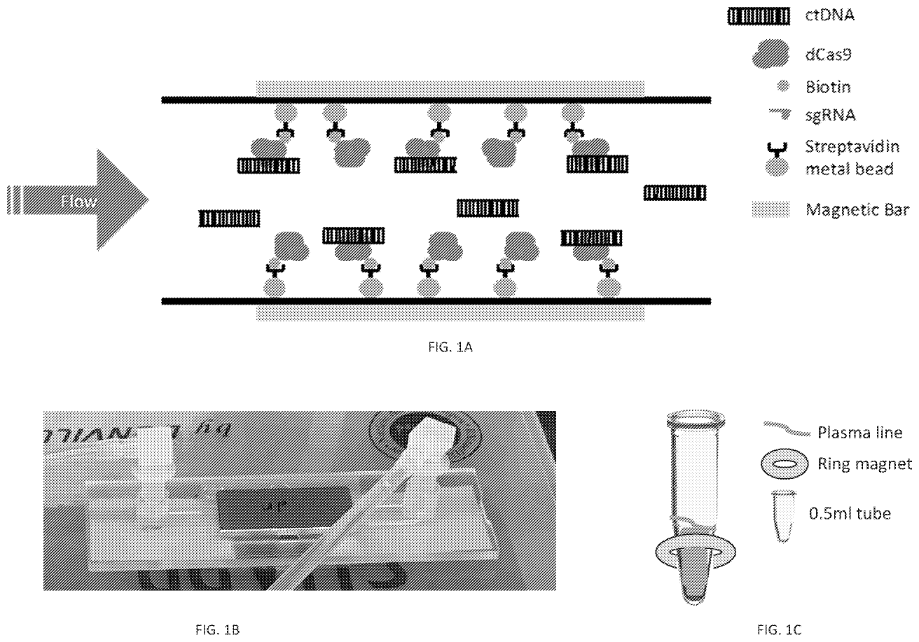

FIG. 2 is schematics of the two-stage allele specific qPCR method.

FIGS. 3A-3E show that the dCas9 capture system has the ability to capture ˜50 copies of BRAF mutation in healthy plasma. cfDNA was extracted from 100 ul of healthy plasma with the BRAF mutation DNA (N=4) and healthy plasma (N=3) (FIG. 3A). The dCas9 capture system captured the BRAF mutation DNA that was spiked into 100 ul of plasma at 25° C. (FIG. 3B; Spike-in N=3; Healthy Plasma N=2). The dCas9 captured system captured the BRAF mutation DNA that was spiked into 100 ul of plasma at 37° C. (FIG. 3C; Spike-in N=3; Healthy Plasma N=3). The dCas9 capture system captured the BRAF mutation DNA that was spiked into 2.5 ml of plasma at 25° C. with a shear force of 6.5 T (FIG. 3D; Spike-in N=3; Healthy Plasma N=3). The dCas9 captured system captured the BRAF mutation DNA that was spiked into 2.5 ml of plasma at 37° C. with the shear force of 5 T (FIG. 3E; Spike-in N=3; Healthy Plasma N=2).

FIGS. 4A-4D show the capture of Staphylococcus aureus at 25° C. (FIG. 4A) and 37° C. (FIG. 4B) and proviral HIV (FIGS. 4C-4D) DNA spiked into plasma at 25° C. (FIG. 4C) and 37° C. (FIG. 4D), as in FIG. 3.

FIG. 5 is a schematic of an exemplary methods for capture of DNA in non-flowing plasma.

FIG. 6 is a schematic of an exemplary methods for capture of DNA in flowing plasma.

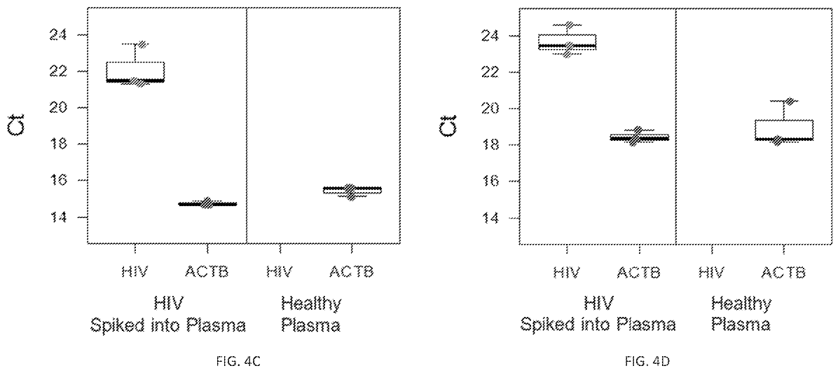

FIG. 7 is an exemplary schematic of methods for capture of multiple circulating free DNAs in a flow chain utilizing two sequential capture devices.

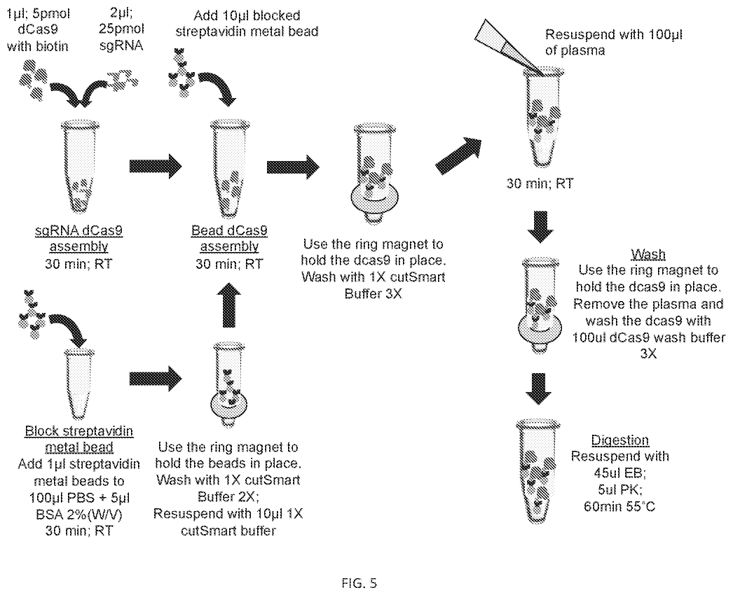

FIG. 8 is a schematic of an exemplary model of the dCas9 capture system.

FIG. 9 is a box plot showing the ability of dCas9 to capture spiked-in BRAF^{Mut} DNA from 100 μL of plasma at 10% MAF at room temperature (25° C.) and body temperature (37° C.); P=t−test.

FIGS. 10A-10D are graphs of the comparison of the ability between the dCas9 capture system and a commercial cfDNA capture kit to capture BRAF^{Mut} from 100 μL of stationary plasma. FIG. 10A is a box plot showing the variation of amplification from the BRAF^{Mut} spiked-in plasma samples at a range of MAFs. FIG. 10B is a box plot showing the percentage of BRAF^{Mut} captured with the dCas9 capture system in comparison to the cfDNA capture kit. FIG. 10C is a box plot showing the fold enrichment of the BRAF^{Mut} in comparison to off-target capture of ACTB. FIG. 10D is bar plots showing the normalized fold recovery of BRAF^{Mut} between the cfDNA capture kit and the dCas9 capture system; P=t-test. MAF=mutant allele fraction.

FIGS. 11A and 11B are graphs showing the comparison of the ability of the dCas9 capture system and a commercially available cfDNA capture kit to capture genomic cfDNA and synthetic DNA from 100 μL of plasma. FIG. 11A is a box plot showing the number of synthetic DNA and genomic cfDNA captured with the cfDNA capture kit and the dCas9 capture system; P=t-test. FIG. 11B is a bar plot showing the normalized fold change between the copies of genomic DNA and synthetic DNA captured by the dCas9 capture system; P=t-test.

FIGS. 12A and 12B are schematics of setups used to flow plasma across the dCas9 capture system. FIG. 12A is a schematic of the laminar flow cell setup. FIG. 12B is a schematic of the turbulent capture chamber setup.

FIGS. 13A and 13B are graphs of the characterization of the dCas9 capture system in laminar flowing unaltered plasma. FIG. 13A is a dot plot showing the ability of the dCas9 to capture BRAF^{Mut} DNA copies using 6 mL of plasma at 10% MAF at a flow rate of 6 or 12 mL/min through flow cells with variable channel heights for 20 min; 6 T and 11 T comparison P=t-test, slope P=coefficient. FIG. 13B is a dot plot showing the ability of the dCas9 to capture BRAF^{Mut} and ACTB DNA copies using 6 mL of plasma at 10% MAF with a flow rate of 6 mL/min and a shear of 3 T at multiple time points; P=coefficient.

FIGS. 14A-14E are graphs of the characterization of the dCas9 capture system in turbulent flowing unaltered plasma. FIG. 14A is a dot plot showing the number of captured BRAF^{Mut} DNA copies using 6 mL of plasma at 10% MAF with a flow rate at 6 mL/min for 20 min using magnets with different surface areas; P=coefficient. FIG. 14B is a dot plot showing the number of captured BRAF^{Mut} DNA copies using 6 mL of plasma at 10% MAF at a flow rate of 6, 12, 26, or 52 mL/min for 20 min; P=coefficient. FIG. 14C is a dot plot showing the number of captured BRAF^{Mut} DNA copies using 6 mL of plasma at 10% MAF with a flow rate of 6 mL/min for 20 min using 1, 2, or 4 times the number of capture molecules; P=coefficient. FIG. 14D is a dot plot showing the number, rate, and reproducibility of DNA copies captured over time with plasma at MAF 10% flowing at 6 mL/min; P=coefficient. FIG. 14E is a dot plot showing the number of BRAF^{Mut} and ACTB DNA copies captured from flowing plasma at 6 mL/min at MAF 10% or MAF 1% at multiple time points. Black bars mark the average number of DNA copies; NA is a nonamplified reaction.

FIGS. 15A and 15B are dot plots showing the number of DNA copies captured by dCas9 at multiple time points in 300 μL of stationary plasma at MAF 10% in the flow cell with a chamber height of 0.8 mm; P=coefficient (FIG. 15A) and 1.5 mL of stationary plasma at MAF 10% in the capture chamber; P=coefficient (FIG. 15B). Bars mark the average number of DNA copies; NA is a non-amplified reaction.

### DETAILED DESCRIPTION

The present disclosure provides methods for the selective capture of cfDNA in plasma from a subject without alteration of the blood plasma, thus allowing the plasma to be returned to the subject. The disclosed methods allow detectable levels of cfDNA to be captured from plasma for cancer detection. Due to the selectivity for cfDNA, plasma samples can be aliquoted for other cancer detection methods (e.g., proteomics, methylation markers, and the like) following cfDNA separation.

Catalytically dead Cas9 (dCas9), guide RNA, and allele-specific quantitative polymerase chain reaction (qPCR) was used to capture and measure the number of captured BRAF^{Mut} DNA copies. dCas9 captured BRAF^{Mut} alleles with equal efficiency at room temperature (25° C.) and body temperature (37° C.). In stationary unaltered plasma, dCas9 was as efficient in capturing BRAF^{Mut} as a commercial cell-free DNA (cfDNA) capture kit. However, in contrast to the cfDNA capture kit, dCas9 enriched BRAF^{Mut} by 1.8-3.3-fold. The capture rate using turbulent flow was greater than that in laminar flow and stationary plasma. With turbulent flow, the number of captured BRAF^{Mut} copies doubles with time (slope=−1.035 Ct) and is highly linear (R²=0.874).

Section headings as used in this section and the entire disclosure herein are merely for organizational purposes and are not intended to be limiting.

### 1. Definitions

The terms “comprise(s),” “include(s),” “having.” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Biomarker,” as used herein, refers to any substance in which its presence, absence, or relative quantity may indicate a particular disease state in a subject. The biomarker includes, but is not limited to, proteins, polypeptides, nucleic acids, small molecules, and the like.

“Biological sample,” as used herein, refers to any suitable sample obtained from any subject, as described here, and includes biological fluids, including, but not limited to, whole blood, serum, plasma, synovial fluid, cerebrospinal fluid, bronchial lavage, ascites fluid, bone marrow aspirate, pleural effusion, urine, as well as tumor tissue or any other bodily constituent or any tissue culture supernatant.

The term “contacting” as used herein refers to bring or put in contact, to be in or come into contact. The term “contact” as used herein refers to a state or condition of touching or of immediate or local proximity.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Methods of Capturing Circulating Free DNA

The present disclosure provides methods of capturing circulating free DNA from a biological sample. The methods comprise forming a capture complex comprising an RNA-guided DNA binding protein, or a functional fragment thereof, bound to a guide RNA configured to at least partially hybridize to a circulating free DNA of interest; incubating the capture complex with the biological sample to form a bound complex comprising the capture complex bound to the circulating free DNA of interest, and removing remaining biological sample from the bound complex.

The biological sample can be any suitable sample obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a nasal swab or brush, or a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. Samples may also be obtained from live or dead organisms or from in vitro cultures. In some embodiment, the biological sample is a fluid, such as human blood (serum or plasma), synovial fluid, cerebrospinal fluid, urine, prostatic fluid, lymph fluid, saliva, and tracheal lavage fluid. In some embodiments, the biological sample comprises blood or blood components. In some embodiments, the biological sample comprises plasma.

The sample can be obtained from the subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

The total volume of the sample can vary depending on the type of sample and the molecule(s) of interest. In some embodiments, the sample have a volume less than 4,000 mL, less than 3,500 mL, less than 3,000 mL, less than 2,500 mL, less than 2,000 mL, less than 1.500 mL, less than 1,000 mL, less than 750 mL, less than 500 mL, less than 250 mL, less than 100 mL, less than 10 mL, less than 1 mL, less than 500 μL, less than 100 μL, less than 50 μL, less than 20 μL, or less than 10 μL.

The sample may be diluted prior to use in the systems and methods disclosed herein. The sample may be diluted about 1-fold, about 2-fold, about 3-fold, about 4-fold, about 5-fold, about 6-fold, about 10-fold, or greater, prior to use. The sample may be concentrated prior to use in the systems and methods disclosed herein. The sample may be concentrated about 1-fold, about 2-fold, about 3-fold, about 4-fold, about 5-fold, about 6-fold, about 10-fold, or greater, prior to use.

Circulating free DNA (cfDNA) refers to extracellular DNA (single-stranded or double-stranded DNA) present in a biological sample. cfDNA may be derived from normal or diseased cells in the subject. The concentration, integrity, genetic, and epigenetic alternations in the cfDNA may suggest pathological conditions of the body, such as infection, inflammation, autoimmune diseases, and cancer. cfDNA comprises various forms of DNA freely circulating in body fluids, including, but not limited to, circulating tumor DNA (cfDNA), circulating cell-free mitochondrial DNA (ccf mtDNA), and cell-free fetal DNA (cffDNA). cfDNA can also be used to describe DNA derived from infectious agents. The present methods and systems are not limited by the type of cfDNA being detected.

In some embodiments, the circulating free DNA is cell free DNA (cfDNA) of microbial (e.g., bacterial, fungal, intracellular, or extracellular parasites) and viral (e.g., bacteriophages, eukaryotic viruses) origin.

In some embodiments, the circulating free DNA is circulating tumor DNA. Circulating tumor DNA (cfDNA) refers to DNA that comes from cancerous cells and tumors. The quantity of cfDNA varies among individuals and depends on the type of tumor, its location, and for cancerous tumors, the cancer stage. cfDNA may be specific for the type of cancer, stage of cancer, or location of cancer, although some cfDNA is found in a variety of different types of cancer. Oftentimes, cfDNA has mutations characteristic of the cancer or tumor cells from which it is derived.

The capture complex comprises an RNA-guided DNA binding protein, or a functional fragment thereof, bound to a guide RNA configured to at least partially hybridize to a circulating free DNA of interest. A functional fragment of an RNA-guided DNA binding protein comprises a region or domain for binding and associating with the guide RNA and, optionally, a region or domain for DNA binding or stabilization of the RNA bound to a corresponding nucleic acid.

In some embodiments, the RNA-guided DNA binding protein is a CRISPR-associated (Cas) protein. The Cas molecule can be from any Type or Class of CRISPR-Cas systems (e.g., Class 1, Class 3, Types I-VI, or any of subtypes thereof). Exemplary Cas proteins include: Cas1, Cas2, Cas3, Cas4, Cas5, Cash, Cas7, Cas8, Cas9, Cas10, Cas12, Cas13, and the like.

In some embodiments, the RNA-guided DNA binding protein is Cas9, or a fragment thereof. The Cas9 protein can be obtained from any suitable microorganism, and a number of bacteria express Cas9 protein orthologs or variants. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases. The Cas 9 protein may be from Streptococcus pyogenes, Staphylococcus aureus (S. aureus), Campylobacter jejuni, Corynebacterium diphtheria, Eubacterium ventriosum, Streptococcus pasteurianus, Lactobacillus farciminis, Sphaerochaeta globus, Azospirillum (strain B510), Gluconacetobacter diazotrophicus, Neisseria cinerea, Roseburia intestinalis, Parvibaculum lavamentivorans, Nitratifractor salsuginis (strain DSM 16511), Campylobacter lari (strain CF89-12), or Streptococcus thermophilus (strain LMD-9). In some embodiments, the Cas9 is from Streptococcus pyogenes or Staphylococcus aureus.

In some embodiments, the Cas9 protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see. e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas9 protein is a catalytically-dead Cas9. Catalytically-dead dCas9 can be obtained, for example, by introducing point mutations (e.g., substitutions, deletions, or additions) in the Cas9 molecule at the DNA-cleavage domain, e.g., the nuclease domain, the RuvC and/or HNH domain. See. e.g., Jinek et al., Science (2012) 337:816-21, incorporated by reference herein in its entirety. For example, introducing two point mutations in the RuvC and HNH domains reduces the Cas9 nuclease activity while retaining the Cas9 RNA and DNA binding activity. For example, Streptococcus pyogenes Cas9 may be rendered catalytically dead by mutations of D10 and at least one of E762, H840, N854, N863, or D986, typically H840 and/or N863A (see. e.g., U.S. Patent Application Publication 2017/0051312, incorporated herein by reference). Mutations in corresponding orthologs are known, such as N580 in Staphylococcus aureus Cas9. Similar mutations can also apply to any other naturally-occurring Cas9 (e.g., Cas9 from other species) or engineered Cas9 molecules. Oftentimes, such mutations cause catalytically-dead Cas9 to possess no more than 3% of the normal nuclease activity.

The gRNA may be a crRNA, crRNA/tracrRNA (or single guide RNA, sgRNA). The terms “gRNA,” “guide RNA,” and “CRISPR guide sequence” may be used interchangeably throughout and refer to a nucleic acid comprising a sequence that determines the binding specificity of the RNA-guided DNA binding protein. A gRNA hybridizes to (complementary to, partially or completely) the target cfDNA.

The gRNA or portion thereof that hybridizes to the cfDNA may be between 15-40 nucleotides in length. In some embodiments, the gRNA sequence that hybridizes to the cfDNA is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology. 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to the cfDNA, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the capture complex is adsorbed to a surface (e.g., microparticle or nanoparticle), or alternatively, tethered to a surface by a linker. In some embodiments, the surface is a particle or head (e.g., microparticle or nanoparticle).

In some embodiments, the capture complex is tethered by a biotin streptavidin linker, such that the RNA-guided DNA binding protein further comprises a biotin tag and the surface comprises streptavidin or similar biotin-binding agent.

The RNA-guided DNA binding protein may comprise an epitope tag (e.g., a FLAG tag, an HA tag, a Myc tag, and the like). In some embodiments, the RNA-guided DNA binding protein comprises a FLAG tag.

The biotin and/or epitope tags may be located at the N-terminus, a C-terminus, or a combination thereof.

As used herein, the term “nanoparticle” refers to small particles having a diameter on the scale of 0.001 μm to 1 μm. As used herein, the term “microparticle” refers to small particles having a diameter on the scale of 1 to 1000 μm. The particles may comprise any material, including metals, semiconductor materials, magnetic materials, and combinations of materials.

In some embodiments, the particle is magnetic. “Magnetic particle,” as used herein, refers to so-called magnetic beads, magnetic microbeads, paramagnetic particles, magnetically attractable particles, magnetic spheres, and magnetically responsive particles. These terms are often used interchangeably throughout the field. As such, “magnetic particle” includes any of the particles capable of being manipulated in a liquid with the application of a magnetic field. The magnetism of the bead may include paramagnetic, superparamagnetic, ferromagnetic, antiferromagnetic, and ferrimagnetic properties.

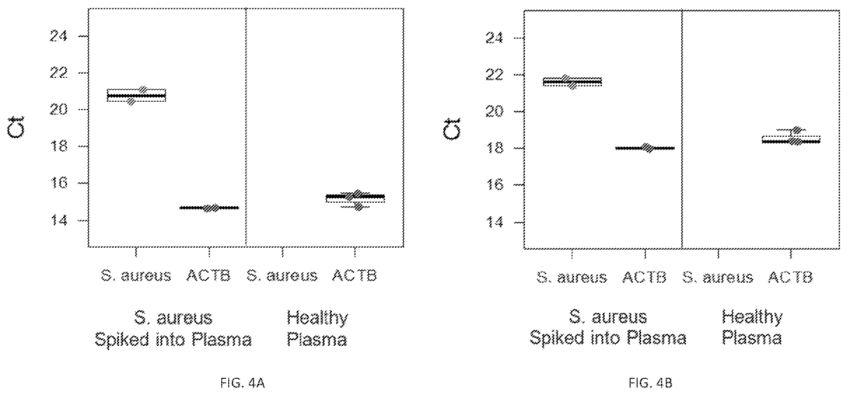

The methods comprise incubating the capture complex with the biological sample to form a bound complex comprising the capture complex bound to the circulating free DNA of interest. Any form of incubation which brings the capture complex in contact with the cfDNA of interest in the biological sample may be utilized with the methods of the present invention. For example, the incubation may take place in a bulk solution, as shown in FIG. 5. The incubation in bulk solution may place the capture complex and the biological sample in any container or reservoir capable of holding the quantity of sample being processed. Containers may include tubes (e.g., microcentrifuge tubes), petri dishes, multi-well plates, and the like.

In some embodiments, the incubating step comprises flowing the biological sample over immobilized capture complexes. The immobilized capture complexes are stationary compared to the direction of flow. Any method of immobilizing the capture complexes, may be utilized with the methods of the present invention. For example, the capture complexes may be immobilized by a covalent linkage to a surface or substrate, the capture complexes may be immobilized magnetically or electrochemically, the capture complexes may be immobilized through immunoprecipitation (e.g., with anti-Cas9, biotin, or epitope tag (e.g., FLAG tag) antibody or binding partner conjugated beads), or the capture complexes may be immobilized physically by partitioning into distinct areas of a surface or substrate. In some embodiments, the capture complexes are magnetically immobilized on the surface of a flow cell or within the fluidic chamber.

The flow cell or fluidic chamber may be any size or shape. The capture complexes may be immobilized on or within any portion of the flow cell or anywhere within the fluidic chamber.

The biological sample may be flowed over the capture complexes at any flow rate or for any amount of time. One of skill in the art can adjust the flow rate and time of incubation between the system and capture complexes based on the binding kinetics of the interaction between the capture complex and the cfDNA of interest. In some embodiments, the sample may be flowed over the capture complexes a single time. Alternatively, in some embodiments, the sample may be reprocessed over the capture complexes more than once to maximize exposure of the sample to the capture complexes. Contact or incubation is desirably maintained for a sufficient period of time to allow for the binding interaction between the capture complex and the cfDNA to occur. In addition, the incubating may be carried out using binding buffer that facilitates the specific binding interaction.

The binding affinity between capture complex and the cfDNA should be sufficient to remain bound under the conditions of the assay, including any wash steps used to remove molecules that are non-specifically bound. In some embodiments, the method comprises one or more wash steps.

The methods disclosed herein may further comprise one or more of: purifying the circulating free DNA of interest; amplifying the circulating free DNA of interest; and sequencing the circulating free DNA of interest.

Purifying the circulating free DNA of interest may comprise separating the cfDNA from the capture complexes (e.g., changing the binding conditions or adding additional competitive binding agent to cause dissociation of cfDNA from capture complexes), digesting the protein components of the capture complexes, cleaning and concentrating the cfDNA, and the like.

In some embodiments of the disclosed methods, one or more nucleic acid amplification reactions may be performed to create multiple copies of the cfDNA of interest. Any suitable amplification method known in the art allowing for sensitive detection of cell free DNA may be used, including by not limited to polymerase chain reaction (PCR), preferably real time PCR, especially probe-based methods such as Taq-Man, Scorpions, Molecular Beacons; and/or isothermal amplification. In some embodiments, the amplification utilizes probes or intercalating dyes for quantitative amplification. In some embodiments, the amplification utilizes barcodes, adapters, or other means for identifying the isolated cfDNA or generating primer binding cites for the cfDNA.

Following or concomitantly with amplification, the cfDNA or amplified nucleic acid may be sequenced. Sequencing can be accomplished using high-throughput systems some of which allow detection of a sequenced nucleotide immediately after or upon its incorporation into a growing strand, e.g., detection of sequence in real time or substantially real time. In some embodiments, sequencing is achieved by next-generation sequencing. In some embodiments, the next-generation sequencing is chosen from the group consisting of pyrosequencing, single-molecule real-time sequencing, sequencing by synthesis, sequencing by ligation (SOLID sequencing) or nanopore sequencing.

In certain embodiments, the method further comprises analyzing at least a portion of the biological sample for the presence or absence of at least one biomarker. The analysis may be done before or after the capture of the cfDNA. The biomarker may be any substance in which its presence, absence, or relative quantity in a subject may indicate a particular disease or stage of disease. Biomarkers have been linked to a number of diseases such as, cancer, diabetes, multiple sclerosis, neurodegenerative disorders, stroke, etc. Examples of commonly measured biomarkers in humans include proteins (e.g., cytokines, metabolic enzymes, cell cycle enzymes, cytoskeletal protein, autoantibodies, growth factors, and neuropeptides), hormones (e.g., steroid hormones, dehydroepiandrosterone (DHEA), estrogen, vasopressin, cholesterol, adrenalin, cortisol, and cortisone), metabolites (e.g., alcohol, lactic acid, lactate, urea, and creatinine), and small molecules (e.g., vitamins, glucose, penicillin, and hydrogen peroxide).

In some embodiments, the methods further comprise returning at least a portion of the biological sample to the subject. For example, once the cfDNA of interest is isolated from the plasma, the remaining plasma can be reintroduced back to the subject using known methods.

### 3. Systems and Devices

Disclosed herein are systems for capturing circulating free DNA from a biological sample. The systems comprise: an RNA-guided DNA binding protein, or a functional fragment thereof and a guide RNA configured to at least partially hybridize to a circulating free DNA of interest. In some embodiments, the system comprises more than one guide RNA configured to bind different circulating free DNA. In some embodiments, the system comprises more than one guide RNA configured to bind a single circulating free DNA. In some embodiments, the system further comprises a solid surface. In certain embodiments, the system further comprises a biological sample. Descriptions of the circulating free DNA, the RNA-guided DNA binding protein, the biological sample, and the solid surface set forth above in connection with the inventive method also are applicable to the disclosed systems.

In some embodiments, the system further comprises a flow cell or fluidic chamber.

The flow cell or fluidic chamber can have any useful structure, such as a well, a channel (e.g., a microchannel), a hole, or a cavity having any cross-section or volume dimension(s). The dimensions may be chosen to maintain a particular volumetric or linear flow rate of a fluid (e.g., biological sample).

In some embodiments, the flow cell or fluidic chamber substrate is fabricated from a material selected from the group consisting of silicon, fused-silica, glass, a polymer, a metal, an elastomer, polydimethylsiloxane, agarose, and a hydrogel, or any combination thereof. In some embodiments, the flow cell or fluidic chamber is fabricated from a material selected from the group consisting of silicon, fused-silica, glass, polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), or any combination of these materials. The flow cell or fluidic chamber may be coated or uncoated for increased flow and decreased non-specific interaction of components of the biological sample. Methods of coating are known in the art.

In some embodiments, the flow cell or fluidic chamber further comprises a transparent window for optical imaging

The flow cell or fluidic chamber may be connected to a fluidic system configured to deliver a biological sample to the flow cell or fluidic chamber, then remove the biological sample from the flow cell or fluidic chamber, and, optionally, re-introduce the biological sample to the flow cell or fluidic chamber. Thus, the flow cell or fluidic chamber may comprise a sample inlet and outlet.

In some embodiments, the fluidic system is further configured to deliver buffer or wash solution to the flow cell or fluidic chamber. The buffer or wash solution may utilize the same or different inlet or outlet to the flow cell or fluidic chamber.

The fluidic system may also include, for example, pumps and valves that are selectively operable for controlling fluid communication within a single flow cell or fluidic chamber or between multiple flow cells or fluidic chambers.

In some embodiments, the immobilization of the capture complexes involves magnetic separation. Thus, in some embodiments, the system further comprises at least one magnet. The at least one magnet may be configured to be arranged above, below, or around at least a portion of the flow cell or fluidic chamber. The magnet may be configured to be actuated, such that the capture complexes are immobilized on or within a surface of the flow cell or within the fluidic chamber when magnet is engaged.

The systems may further comprise reagents for amplifying and/or sequencing the cfDNA. Many such reagents are known in the art and commercially available. Examples of suitable reagents include conventional reagents employed in nucleic acid amplification reactions, such as, for example, one or more enzymes having polymerase activity, enzyme cofactors (such as magnesium or nicotinamide adenine dinucleotide (NAD)), salts, buffers, deoxyribonucleotide, or ribonucleotide triphosphates (dNTPs/rNTPs; for example, deoxyadenosine triphosphate, deoxyguanosine triphosphate, deoxycytidine triphosphate, and deoxythymidine triphosphate) blocking agents, labeling agents, and the like. Other reagents used in amplification reactions include nicking enzymes, single-strand binding proteins, helicases, resolvases, and the like.

One or more of the components of the system may be provided as a composition. The compositions may further comprise carriers. Carriers may include any and all solvents, dispersion media, antibacterial and antifungal agents, isotonic and absorption delaying agents. Some examples of materials which can serve as carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; tale; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. The compositions of the present invention and methods for their preparation will be readily apparent to those skilled in the art. Techniques and formulations may be found, for example, in Remington's Pharmaceutical Sciences, 19th Edition (Mack Publishing Company. 1995).

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

Further provided herein are devices comprising at least one system as described above. In some embodiments, the device comprises multiple systems directed to different circulating free DNAs. In some embodiments, the device is a microfluidic device.

The device may be pre-loaded with any useful component of the system. For example, the device may be preloaded with a flow cell or fluidic chamber containing immobilized capture complexes. Alternatively, the device may allow pre-loaded flow cells or fluidic chambers to be changed out from one use to the next. As such, the present disclosure provides for pre-loaded flow cells or fluidic chambers comprising an immobilized capture complex, or a portion thereof (e.g., immobilized streptavidin beads.

The device may allow for multiplexing, e.g., allow for loading of multiple flow cells or fluidic chambers in the sample path.

Furthermore, the devices and systems disclosed herein can be integrated with other devices to allow multistep processes. For example, the devices or system can be integrated with other analysis devices for detection of other biomarkers and/or sequencing of the cfDNA.

In many embodiments, the devices and systems comprise a computer (or processor) and computer-readable media for providing a user interface as well as manual, semi-automated, or fully-automated control of all system functions, e.g., control of the fluidics system (e.g., volumetric fluid flow rates, fluid flow velocities, the timing and duration for sample and bead addition, reagent addition, and rinse steps), the temperature control system (e.g., specifying temperature set point(s)), and integration with other equipment or devices. In some embodiments, the computer or processor may be an integrated component of the device. In some embodiments, the computer or processor may be a stand-alone module, for example, a personal computer or laptop computer.

It is understood that the disclosed systems or devices may be employed to carry out the disclosed methods.

### 4. Kits

Also provided herein are kits for performing the above-described methods. The kit may include the components of a system or device as described herein.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates, flasks).

The kit can further contain control samples to be analyzed separately from, or concurrently with, the sample from the subject as described above. Optionally, the kit includes quality control components (for example, sensitivity panels, calibrators, and positive controls).

If desired, the kit can further comprise one or more components, alone or in further combination with instructions, for assaying the test sample for another analyte, which can be a biomarker

The kits provided herein are in suitable packaging. Suitable packaging includes, but is not limited to, vials, bottles, jars, flexible packaging, and the like. Individual member components of the kits may be physically packaged together or separately.

### 5. Examples

### Materials and Methods

Magnets For all washing and non-flow dCas9 capturing steps, a countersunk ring magnet (K&J magnet RX033CS-P-N52) was used to pull-down the dCas9 attached metal beads in a 0.5 ml PCR tube. For the flow capture experiments, two bar magnets (K&J magnet BX081-N52), were used on a p-slide luer (ibidi 80191).

BRAF mutation spike-in BRAF^{Mut} and synthetic BRAF^{Wt} duplexed DNA modified with phosphate 3′ ends (IDT) was diluted with TE buffer (IDT 11-05-01-05), mixed with 5 ng/ul RNA carrier (Qiagen 142342312), and was spiked into healthy plasma. The plasma used in this study was pooled plasma from gender-unspecific healthy individuals purchased from BiolVT (K2EDTA plasma, BiolVT). To compare the efficiency of capture of cfDNA from plasma, the Quick-cfDNA serum and plasma kit (Zymo, D4076) was used. The mutant allele fraction was calculated as the number of BRAFMut DNA copies divided by the number of ACTB DNA copies.

Blocking Beads For each reaction, 1 or 2 μl of metal beads (Dynabeads-Streptavidin M280) were blocked by incubating in 100 μl of Bead Blocking buffer (95 μl PBS and 0.1% (w/v) BSA (NEB B9000S) for 30 min at room temperature. A countersunk ring magnet (K&J magnet, RX033CS-P-N52) was used to pull down the metal beads. The blocked beads were then washed 2× with 100 ul 1× cutSmart buffer (NEB B7204S) and resuspended with 10 μl 1× cutSmart buffer per reaction.

Assembling RNPs For each reaction. 5 pmol of dCas9-3×FLAGTM Biotin Protein (MilliporeSigma) was incubated with 40-50 pmol sgRNA (IDT) for 30 min at room temperature.

RNPs Beads Assembling The 10 μl of blocked beads were mixed with the assembled RNPs and was incubated for 30 min at room temperature. The RNP assembled bead mixture was pulled down with a magnet and washed three times with 100 ul 1× cutSmart buffer and resuspended with 100-300 ul 1× cutSmart buffer. After the ring or bar magnet was put in place, the 1× cutSmart buffer was removed and the plasma was added to the system. The ring magnet was then removed and the plasma and RNPs were mixed by taping the PCR tube.

dCas9 capture The spiked-in plasma mixture was incubated with the RNPs Beads for 1-hour at room temperature or 37° C. The RNPs and captured DNA were placed on the ring magnet, the plasma was removed, and the beads were washed 3 times with filtered sterilized Cas9 washing buffer (50 mM Tris (pH8), 150 mM NaCl, and 0.05% tween 20). The washed Cas9 was resuspended in 45 μl EB buffer (Zymo D3004-4-50) and digested with 5 μl PK (Invitrogen 25530049) at 55° C. for 1 hour.

Digested dcas9 DNA clean-up DNA clean & concentrator-5 (Zymo D4013) was used to clean-up the digested cfDNA and was eluted with 6 ul of EB buffer. This DNA clean and concentrator-5 (Zymo, D4013) kit was also used to concentrate the cfDNA extracted using the Quick-cfDNA serum and plasma kit. Each reaction was eluted with 6 μL of EB buffer.

Allele Specific Duplex PCR The allele specific duplex PCR assay procedure consisted of two sequential PCR reactions. In Step 1 PCR, (multiplexed generation of tailed end amplicons). 5 μl of cfDNA was added to 15 μl of reaction buffer [1.25 mM deoxynucleotide triphosphates, 16.6 mM (NH₄)₂SO₄, 67 mM Tris (pH 8.8), 6.7 mM MgCl₂, 10 mM β-mercaptoethanol, 0.1% DMSO, 5 unit of Platinum Taq (Invitrogen) and 400 nM each of the forward and reverse primers]. Conditions used for this PCR step were: 95° C. for 5 min, followed by 20 cycles of 95° C. for 30 s, 65-68° C. for 45 s, and 72° C. for 45 s, with a final extension cycle of 72° C. for 7 min. The PCR products were diluted to 100 μl with reaction buffer and stored at −20° C.

For Step 2 PCR (Quantitative multiplexed PCR of amplicons), 4 μl of the diluted DNA was added to 16 μl qPCR reaction buffer [16.6 mM (NH₄)₂SO₄, 67.0 mM Tris (pH 8.8). 6.7 mM MgCl₂, 10.0 mM β-mercaptoethanol, 0.1% DMSO, 200 μM deoxynucleotide triphosphates, 1.25 units immolase DNA polymerase (bioline), 50 ug/ml tRNA (Invitrogen) and 300 nM ROX (Invitrogen)], 700 nM each of primers (forward and reverse) and 200 nM labeled probe (Applied Biosystems). The reaction was carried out in a 96-well reaction plate in a 7500 Fast Real-Time PCR (Applied Biosystems). The reaction conditions were: 95° C. for 10 min, followed by 40 cycles of 95° C. for 15 s and 56° C. for 1 min.

### Capture of DNA in Non-Flowing Plasma

To form the sgRNA and dCas9 complex, dCas9 (1 μl of 5 pmol solution) was mixed with a corresponding sgRNA (2 μl 25 pmol) and incubated at room temperature for 30 min.

The streptavidin metal beads were blocked by incubating with a mixture of 100 μl PBS and 5 μl BSA (2% w/v) and washed 2 times with 100 ul of 1× cutSmart buffer prior to resuspension in 10 μl of 1× cutSmart buffer.

To form the dCas9 bead complex, blocked beads (10 μl) were mixed with the assembled sgRNA dCas9 complex and incubated at room temperature for 30 minutes followed by washing with 3 times with 1× cutSmart buffer (New England Biolabs).

A quantity of the dCas9 bead complex was incubated with a plasma sampled for 30 minutes at room temperature. The beads with bound cargo were sequestered from remaining plasma sample using a ring magnet. After removal of the plasma, the separated beads were washed 3 times with 100 μl dCas9 wash buffer before resuspension in EB buffer (45 μl).

For digestion, PK (5 μl) was added and the solution was incubated at 55° C. for 60 min. The remaining DNA was clean and concentrated with DNA Clean & Concentrator-5 Kit (Zymo Research) according to manufacturer's protocol prior to PCR.

### Capture of DNA in Flowing Plasma

The sgRNA and dCas9 complex and the dCas9 bead complexes were formed as described in the previous capture method. The dCas9 bead complexes were resuspended in cutSmart buffer (100 μl) and added to the p-Slide 10.2 Luer. Using bar magnets, the dCas9 bead complex were tethered to the interior surface of the chamber and the buffer was removed. Plasma, at least 2.5 ml, was flowed through the chamber with a peristaltic pump at 11.9 ml/min (shear 55 T) for 30 min at room temperature. Any remaining plasma was removed from the μ-Slide 10.2 Luer chamber prior to removal of the magnets and resuspension of the dCas9 bead complex in 100 μl of 1× cutSmart buffer.

Following removal of the dCas9 bead complexes from the μ-Slide 10.2 Luer they were washed, digested, and prepared for PCR as described in the previous capture method.

### Example 1

### Mutant BRAF DNA Capture

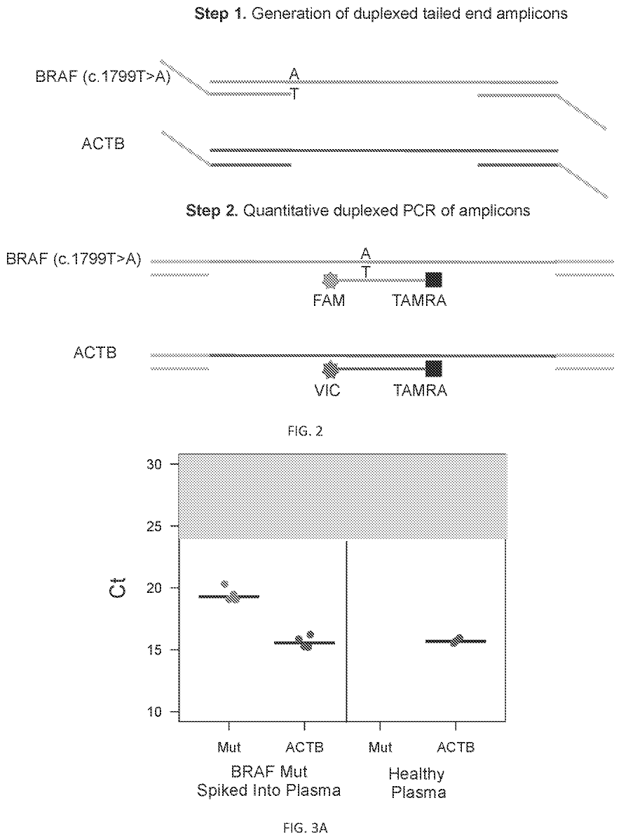

An exemplary version of the disclosed system was used to capture BRAF (T1799A) DNA. BRAF mutant DNA was spiked into plasma. To avoid endonuclease degradation, a 90 mer duplexed oligonucleotide sequenced with a 3′ phosphorylation modification was synthesized (Table 1). A two-stage allele specific qPCR was used to measure that amount of on-target and off-target DNA binding (Table 1 and FIG. 2).

The first step of the qPCR amplified two markers and actin control (ACTB) control using a single aliquot of DNA in one well, with the forward primer 3′ end specific for the T1799A BRAF mutation. The 5′ end of the forward and reverse primers for the BRAF mutation and ACTB have synthetic tails. In the second step of PCR, primers that are complimentary to the synthetic tails were used, along with marker-specific TaqMan probes, each with different indicated fluorescent tags. Both markers are amplified in a single real-time PCR reaction.

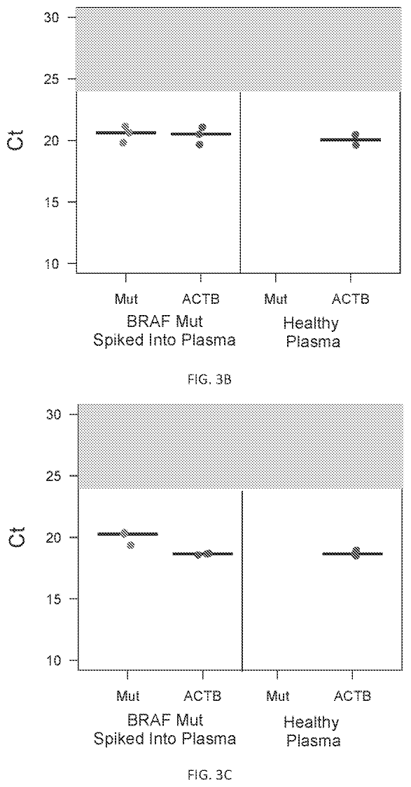

Approximately fifty copies of BRAF mutant DNA were spiked into 100 μl of healthy plasma. ˜8% BRAF mutation DNA. After the 100 μl of the simulated cancer patient plasma sample DNA was extracted, the average Ct value for the BRAF mutant DNA Ct was 19.5 while the average ACTB Ct was 15.63 (FIG. 3A). Since 100 μl of healthy plasma contains ˜600 copies of gDNA, the average copy number of BRAF mutation DNA spiked into the healthy plasma was determined to be 42 copies. Because a Ct of 24 is the equivalent of ˜1 copy of DNA, a Ct of 24 was selected as the maximum threshold for measuring copy numbers for both ACTB and BRAF mutation DNA. Using the 24 Ct threshold, the healthy plasma did not show amplification of BRAF mutation DNA for any of the healthy plasma controls tested (FIGS. 3A-3E).

To test if an exemplary dCas9 system can enrich for the mutant DNA, 100 ul of plasma was spiked with ˜50 copies of mutant DNA and the dCas9 was incubated in the plasma for 1 hr at room temperature (25° C.), using the Cts calculated in FIG. 3A, on average dCas9 captured 48% of the total amount of mutant BRAF DNA (average Ct 20.5) (FIG. 3B). The dCas9 also captured 5% of the ACTB DNA (˜30 copies) (average Ct 20.1) from the healthy plasma. This shows that a dCas9 capture system enriched the BRAF mutation DNA 9.7 fold more than the non-specific ACTB DNA. At 37° C., on average, the dCas9 system captured greater numbers of both mutate DNA (70%; average Ct 20.0) and ACTB DNA (13%; average Ct 18.7) (FIG. 3C). The enrichment at 37° C. was 5.5 fold

Next, a peristaltic pump was used to flow 2.5 ml of plasma with ˜1250 copies of spiked-in mutant DNA, ˜8% BRAF mutation DNA, across the dCas9 capture system. After the plasma was flowed past the dCas9 system with a shear force of 6.5 T at 25° C. for 1 hour, the average amount of mutant DNA capture was 20% (average Ct 21.8) of the original copies while only 3% (average Ct 20.9) of the ACTB DNA was captured (FIG. 3D). This setup showed that the enrichment of the BRAF mutation DNA was 7.2 more than the ACTB DNA.

Finally, 2.5 ml of plasma with ˜1250 copies of spiked-in mutant DNA was flowed past the dCas9 system with a shear force of 5.0 T at 37° C. for 1 hour was analyzed. The average amount of mutant DNA captured was 16% (average Ct 22.1) of the original copies while 8% (average Ct 19.3) of ACTB DNA was captured (FIG. 3E). The enrichment of the BRAF mutation DNA was 1.9 more than the ACTB DNA.

### Example 2

### Non-Tumor DNA

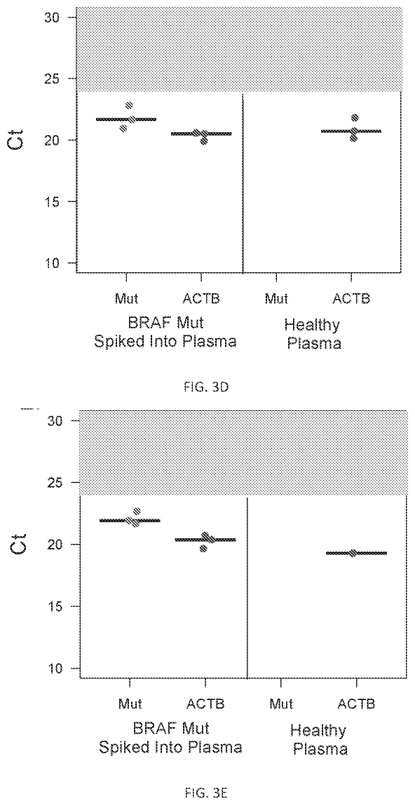

Approximately 50 copies of 16S from S. aureus DNA was spiked into 100 ul of healthy plasma, ˜8% spike-in DNA. After the 100 ul of the simulated patient plasma sample DNA was extracted using a cfDNA extraction kit, the average Ct value for the S. aureus was 20.76 Ct while the average ACTB Ct was 15.14 (FIG. 4A). Using this 24 Ct threshold, the healthy plasma did not show amplification of S. aureus DNA for any of the healthy plasma controls tested (FIGS. 4A and 4B).

To test if the dCas9 system can enrich for the S. aureus DNA, 100 ul of plasma was spiked with ˜50 copies of DNA and incubated the dCas9 in the plasma for 1 hr at room temperature (25° C.). Using the Cts calculated above, on average the dCas9 captured 56% of the total amount of S. aureus DNA (average Ct 21.59)(FIG. 4B). The dCas9 also captured 9% of the ACTB DNA (average Ct 18.68) from the healthy plasma. This shows that the dCas9 capture system can enrichment the S. aureus DNA 6.5 fold more than the non-specific ACTB DNA.

To simulate a cancer patient plasma sample, ˜50 copies of proviral HIV DNA was spiked in into 100 ul of healthy plasma, ˜8% spike-in DNA. After the 100 ul of the simulated patient plasma sample DNA was extracted using a cfDNA extraction kit, the average Ct value for the proviral HIV DNA was 22.08 Ct while the average ACTB Ct was 15.42 (FIG. 4C). Using this 24 Ct threshold, the healthy plasma did not show amplification of proviral HIV DNA for any of the healthy plasma controls tested (FIGS. 4C and 4D).

To test if the dCas9 system can enrich for the proviral HIV DNA, 100 ul of plasma was spiked with ˜50 copies of DNA and incubated the dCas9 in the plasma for 1 hr at room temperature (25° C.). Using the Cts calculated previously, on average the dCas9 captured 33% of the total amount of proviral HIV DNA (average Ct 23.66)(FIG. 4D). The dCas9 also captured 9% of the ACTB DNA (average Ct 18.95) from the healthy plasma. This shows that the dCas9 capture system can enrichment the proviral HIV DNA 3.9 fold more than the non-specific ACTB DNA.

### Example 3

### cfDNA Capture in Clinical Applications

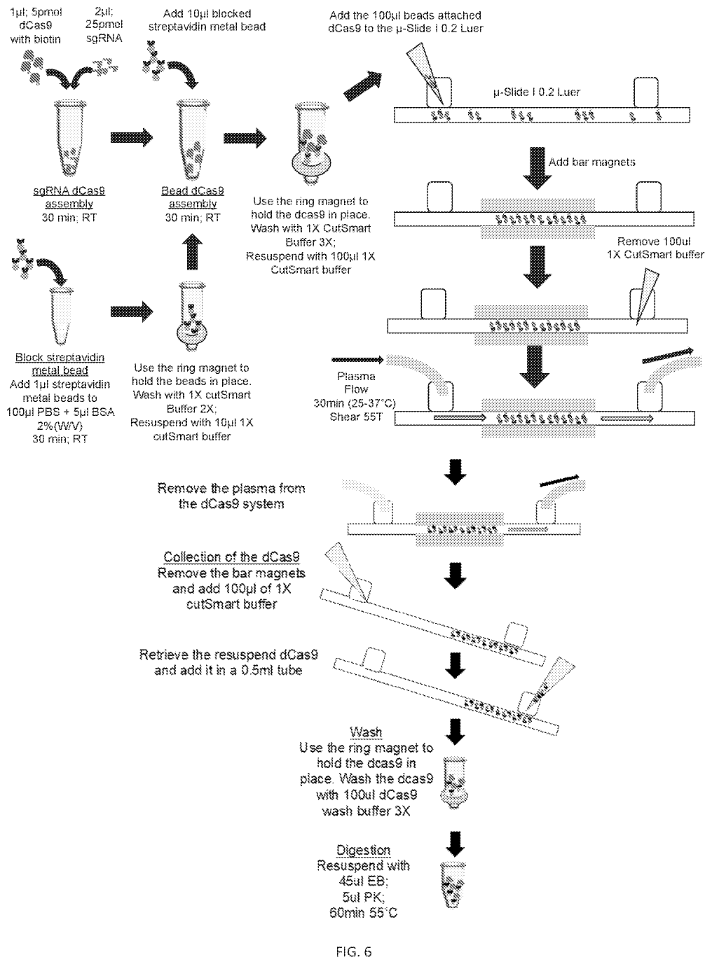

An illustration showing the envisioned clinical application of the dCas9 capture system is shown in FIG. 8.

Conditions were established for a qPCR method to measure the cfDNA captured by the dCas9. While the sgRNA drives the specificity for DNA binding for the dCas9 protein, it is well-understood that off-target capture can occur. To investigate effects caused by off-target binding, both on-target, BRAF^{Mut}, and off-target DNA capture were measured. Since allele-specific PCR was being used to measure BRAF^{Mut}, BRAF^{Wt} was not used as the off-target control. To investigate if ACTB could be used as an off-target DNA control, BRAF^{Wt} and ACTB were subjected to duplexed PCR using cfDNA extracted from 100 μL of pooled plasma from healthy individuals. 100 μL of plasma contained between 398 and 485 copies of genomic cfDNA, and there was no difference in the amplification efficiency between BRAF^{Wt} and ACTB; t-test P=0.748. On the basis of this result, ACTB was selected as the off-target capture control.

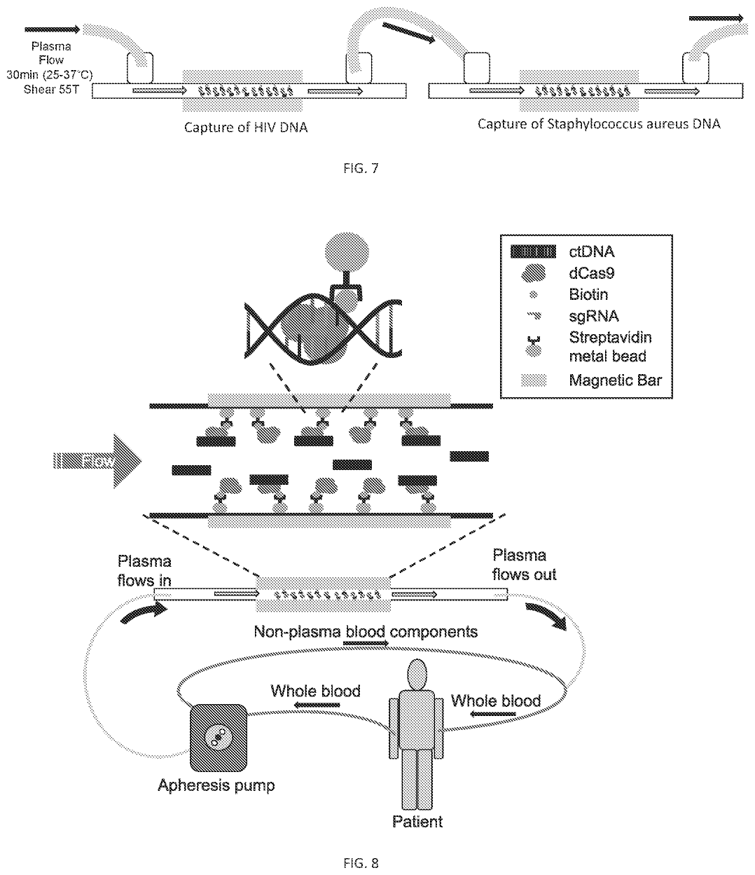

To determine the optimal capture temperature for dCas9, the ability to capture BRAF^{Mut} spiked into 100 μL of healthy plasma at mutant allele fraction (MAF) 10%, at both body temperature (37° C.) and room temperature (25° C.) was tested. No significant difference was found between the number of BRAF^{Mut} DNA copies captured between these two temperatures (t-test P=0.642) (FIG. 9). Since the capture rate was equivalent between 37° C. and room temperature (25° C.), all further capture experiments were performed at room temperature.

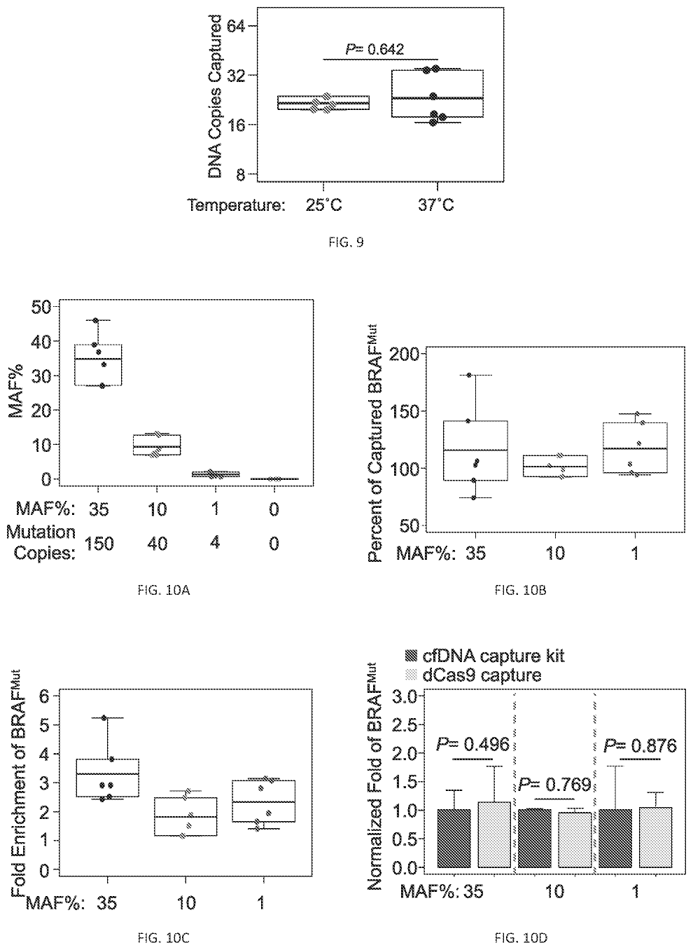

The ability of the dCas9 and the Zymo Quick-cfDNA Serum & Plasma Kit to capture cfDNA from 100 μL of plasma spiked-in with BRAF^{Mut} at a range of MAFs was compared. This MAF range included plasma samples at MAF 35% (150 mutated DNA copies). MAF 10% (40 mutated DNA copies), and MAF 1% (4 mutated DNA copies) (FIG. 10A). On average, the dCas9 capture system captured between 101 and 117% of the BRAF^{Mut} copies in comparison to the cfDNA capture kit (FIG. 10B). Furthermore, the dCas9 system enriched the BRAF^{Mut} on average between 1.8- and 3.3-fold higher in comparison to ACTB (t-test P<0.05) (FIG. 10C). Overall, no statistical difference was found between the number of BRAF^{Mut} DNA copies captured using the cfDNA capture kit in comparison to the dCas9 capture system at all tested MAFs (t-test P>0.05) (FIG. 10D).

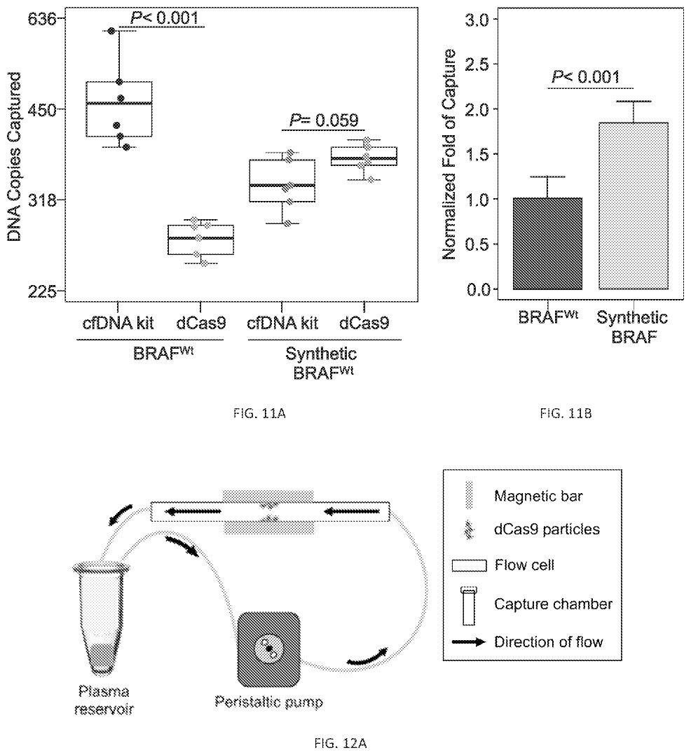

To determine if the dCas9 can capture genomic cfDNA from human plasma samples and to investigate the quality of the spiked-in plasma surrogates, the ability of the dCas9 capture system to capture genomic wild-type BRAF cfDNA from plasma to its ability to capture synthetic BRAF^{Wt} DNA spiked into plasma was compared. Unlike genomic cfDNA, synthetic DNA is uniform in size and is unbound to proteins. To distinguish between the synthetic and genomic BRAF^{Wt}, nonhuman DNA sequences were added to the 5′ and 3′ ends of the synthetic DNA outside the sgRNA targeted DNA sequence (Table 1). After spiking in the synthetic DNA at copy numbers similar to the number of genomic BRAF^{Wt} cfDNA copies in 100 μL of plasma, it was found that the dCas9 capture system captured 62% of the genomic DNA compared to the cfDNA capture kit (t-test P<0.001) (FIG. 11A). Furthermore, the dCas9 capture system captured 0.85-fold more synthetic BRAF^{Wt} DNA than the genomic BRAF^{Wt} cfDNA (t-test P<0.001) (FIG. 11B). As previously noted, a significant difference was not found between the number of synthetic BRAF^{Wt} DNA captured with the dCas9 capture system in comparison to the cfDNA capture kit (t-test P=0.059) (FIG. 11A).

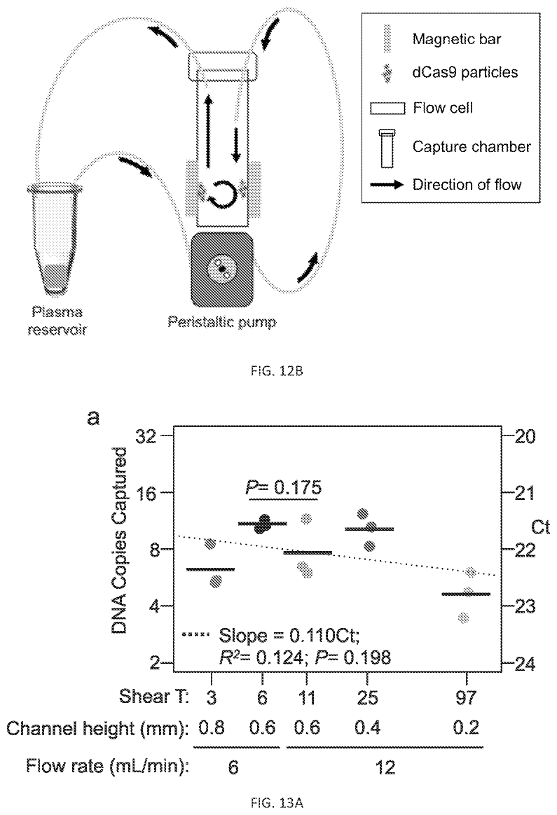

To investigate if the dCas9 capture system could capture BRAF^{Mut} in flowing unaltered plasma, the dCas9 capture system into a flow cell or into a simple capture chamber in order to characterize the capture ability using laminar and turbulent flows (FIGS. 12A and 12B). A peristaltic pump was used to flow the plasma across the dCas9 capture system within the flow cell or capture chamber. To pump a larger volume of plasma across the dCas9 capture system than the capacity of the flow cell or capture chamber, a 15 mL reservoir was added to the flow systems.

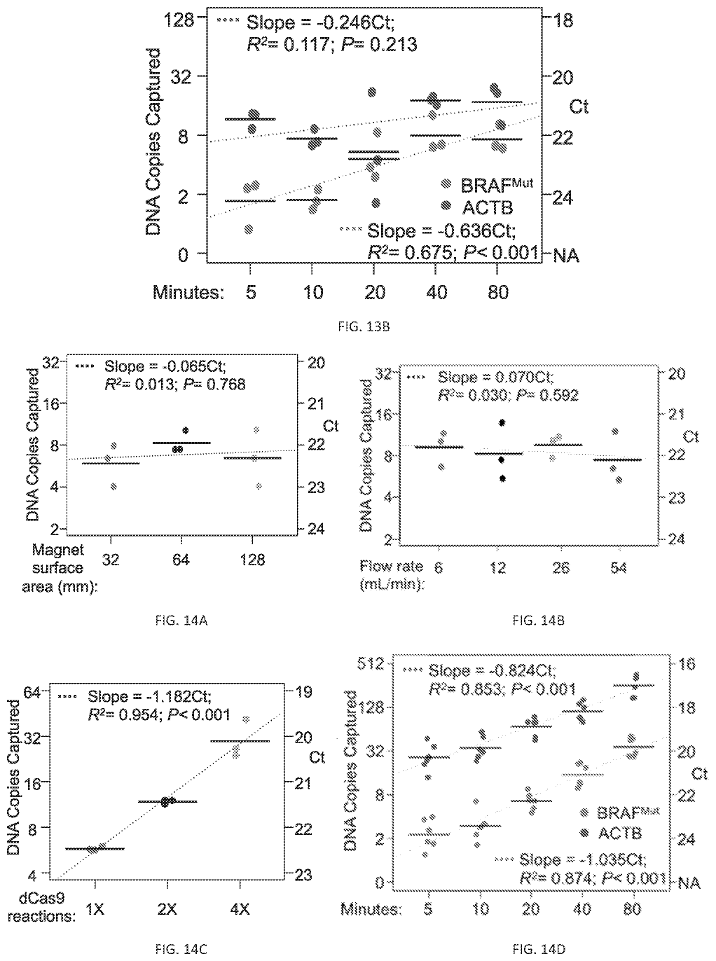

To test if the shear force has an effect on the dCas9 capture, plasma at MAF 10% was pumped through the commercial flow cells with different chamber heights (0.8, 0.6, 0.4, or 0.2 mm) for 20 min at a flow rate of 6 or 12 mL/min depending on the chamber height. No correlation was found between the number of BRAF^{Mut} DNA copies captured and the five shear forces that were tested (slope=0.11 Ct; R²=0.124; coefficient P=0.198) (FIG. 13A). Furthermore, no significant difference was found in the number of BRAF^{Mut} copies captured when using a flow rate of 6 compared to 12 mL/min for 20 min (t-test P=0.175) (FIG. 13A). Next, the number of BRAF^{Mut} and ACTB DNA copies captured was measured with the dCas9 from plasma at 10% MAF using a laminar flow at a rate of 6 mL/min and a shear of 3 T at multiple time points. In laminar flow, the dCas9 captured BRAF^{Mut} DNA copies with poor linearity, and the number of DNA copies captured did not double with time (slope=−0.636 Ct; R²=0.675; coefficient P<0.001) (FIG. 13B).

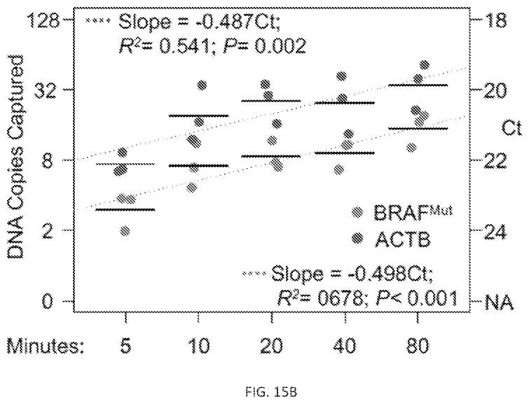

To compare the ability of dCas9 to capture BRAF^{Mut} DNA using flowing and stationary plasma, the dCas9 was incubated in a flow cell filled with stationary plasma (300 μL) at MAF 10% for various lengths of time. With stationary plasma, the rate of BRAF^{Mut} capture was similar to that of flowing plasma (slope=−0.67 Ct) but had greater linearity (R²=0.822; coefficient P<0.001) (FIG. 15A). Furthermore, no difference in the number of BRAF^{Mut} DNA copies captured was found in flowing plasma in comparison to 300 μL of stationary plasma in the flow cell at the 80 min time point (t-test P=0.08).

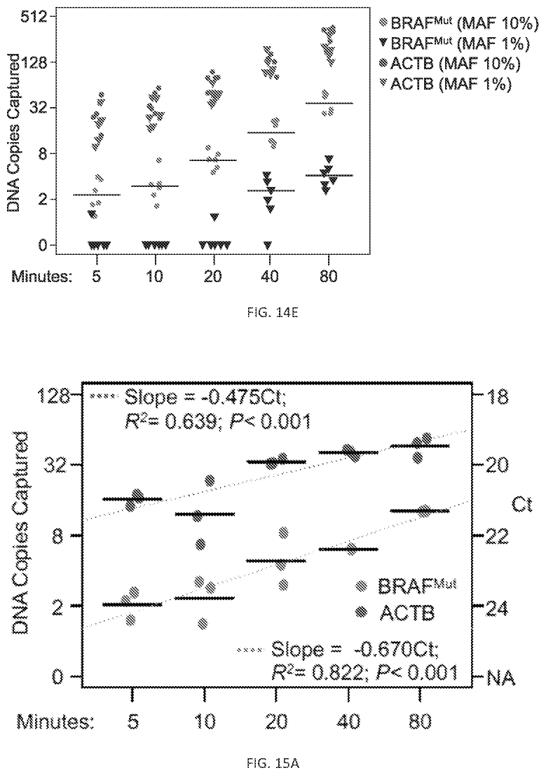

Next, the ability of the dCas9 to capture BRAF^{Mut} DNA in turbulent flow was characterized. The surface area of the magnet (32, 64, or 128 mm) used to keep the dCas9 beads stationary in the flow chamber was tested to determine if it had an effect on the number of BRAF^{Mut} DNA copies captured using plasma at 10% MAF at a flow rate of 6 mL/min for 20 min. No correlation was found between the number of BRAF^{Mut} DNA copies captured and the surface area of the magnet (slope=−0.065 Ct; R²=0.013; coefficient P=0.768) (FIG. 14A). Next, the flow rate (6, 12, 26, or 52 mL/min) of 6 mL of plasma at MAF 10% for 20 min was tested to determine if it correlated with the number of BRAF^{Mut} DNA copies captured. Similar to laminar flow, there was no evidence that the flow rate of the plasma had an effect on the number of BRAF^{Mut} DNA copies captured (slope=0.07 Ct; R²=0.030; coefficient P=0.592) (FIG. 14B). The number of captured molecules was investigated to determine if it correlated with the number of BRAF^{Mut} DNA copies captured from 6 mL of flowing plasma at 10% MAF with a flow rate of 6 mL/min for 20 min. The number of captured BRAF^{Mut} DNA copies doubled as the number of dCas9 captured molecules doubled (slope=−1.182 Ct; R²=0.954; coefficient P<0.001) (FIG. 14C). The number of BRAF^{Mut} and ACTB DNA copies captured by the dCas9 capture system from plasma at 10% MAF using a flow rate of 6 mL/min at multiple time points was measured. With turbulent flow, the number of captured BRAF^{Mut} copies doubled with time (slope=−1.035 Ct) and was highly linear (R²=0.874); coefficient P<0.001 (FIGS. 14D and 14E). In plasma at MAF 10% and the flow rate of 6 mL/min, the dCas9 capture system captured 0.4 BRAF^{Mut} copies per minute. Since the entire 6 mL volume of the plasma passed through the capture device every minute, the MAF % was calculated to decrease by 0.001% per minute. However, since the slope is close to ˜1.0 Ct and the R² value was very high, there is little evidence that (his small decrease of MAF significantly affected the number of BRAF captured in this plasma flowing experiment.

To investigate if BRAF^{Mut} could be captured from plasma at a lower MAF, the flow capture experiment was repeated using plasma at MAF 1%. The dCas9 capture system could reproducibly capture BRAF^{Mut} at MAF 1% at the 40 mini time point (five of six replicates showed BRAF^{Mut} capture) and at the 80 min time point (six of six replicates showed BRAF^{Mut} capture) (FIG. 14E). Furthermore, at the 80 min time point, the average fold difference between the number of BRAF^{Mut} DNA copies captured from the plasma at MAF 10% and MAF 1% was 8.90-fold (t-test P<0.001).

Finally, to investigate if dCas9 could capture more BRAF^{Mut} using flowing plasma than in stationary plasma, the dCas9 was incubated in a capture chamber full of stationary plasma (1.5 mL) at MAF 10% for various lengths of time. With the stationary plasma, the rate of BRAF^{Mut} capture was half that of the flowing plasma (slope=−0.498 Ct) and was also less linear (R²=0.678; coefficient P<0.001) (FIG. 15B). Furthermore, at the 80 min time point, the dCas9 in the stationary plasma captured 3.6-fold less BRAF^{Mut} DNA copies than with the turbulent flowing plasma (t-test P=0.005).

TABLE 1
sgRNA, DNA, Primers, and Probes

| | | Location |
|---|---|---|
| Name | Sequence 5′ to 3′ | (hg38) |
| sgRNA Mut | TAGCTACAGAGAAATCTCGA (SEQ ID NO: 1) | chr7: |
| BRAF | | 140753323- |
| | | 149753343 |
| Mut BRAF | GACCCACTCCATCGAGATTTCTCTGTAGCTAGACCAAAAT | chr7: |
| (T1799A) | CACCTATTTTTACTGTGAGGTCTTCATGAAGAAATATATC | 140753315- |
| | TGAGGTGTAG′P (SEQ ID NO: 2) | 140753404 |
| Mut BRAF | CATCTGCCTGAGTATGAAGACCCACTCCATCGAGATTTCT | chr7: |
| Forward | (SEQ ID NO: 3) | 140753315- |
| | | 140753404 |
| Mut BRAF | GTTCAGTGTAGCTTACTGTATGAAGACCTCACAGTAAAA | |
| Reverse | ATAGGTG (SEQ ID NO: 4) | |
| Mut_BRAF | TAGCTACAGAGAAATC (SEQ ID NO: 5) | |
| Probe | | |
| sgRNA Wt | TAGCTACAGTGAAATCTCGA (SEQ ID NO: 6) | |
| BRAF | | |
| Synthetic | GTCCCAGGAGAACGAAGTTGCCGACTCCATCGAGATTTC | |
| Wt BRAF | ACTGTAGCTAGACCAAAATTATTGTAGAGCAGTGCTGTC | |
| | ACAAGCCT′P (SEQ ID NO: 7) | |
| Wt BRAF | GTTGTCGATGCTATCTCTAAATGGATCCAGACAACTGTTC | chr7: |
| Forward | AAAC (SEQ ID NO: 8) | 140753283- |
| | | 140753381 |
| Wt BRAF | GTTCAGTGTAGCTTACTGTATGAAGACCTCACAGTAAAA | |
| Reverse | ATAGGTG (SEQ ID NO: 9) | |
| Wt BRAF | CTAGCTACAGTGAAATC (SEQ ID NO: 10) | |
| Probe | | |
| Synthetic | CATCTGCCTGAGTATGAAGTCCCAGGAGAACGAAGTTGC | |
| Wt BRAF | C (SEQ ID NO: 11) | |
| Forward | | |
| Synthetic | CTTGATGTCAGTTAGGAAGGCTTGTGACAGCACTGCTCTA | |
| Wt BRAF | CAATA (SEQ ID NO: 12) | |
| Reverse | | |
| ACTB | CATCTGCCTGAGTATGAAAGTCTCCACTCACCCAGGAA | chr7: |
| Forward | (SEQ ID NO: 13) | 5528262- |
| | | 5528346 |
| ACTB | GTTGTCGATGCTATCTCTAGAGCTACGAGCTGCCTGAC | |
| Reverse | (SEQ ID NO: 14) | |
| ACTB | CCCTGAGGCACTCTTCCAG (SEQ ID NO: 15) | |
| Probe | | |
| Synthetic | CATCTGCCTGAGTATGAA (SEQ ID NO: 16) | |
| Tail 1 | | |
| Synthetic | GTTGTCGATGCTATCTCT (SEQ ID NO: 17) | |
| Tail 2 | | |
| Synthetic | GTTCAGTGTAGCTTACTGTAT (SEQ ID NO: 18 ) | |
| Tail 3 | | |

Underline: Human genomic sequences

TABLE 2
sgRNA, DNA, Primers, and Probes for non-tumor DNA

| Name | Sequence 5′ to 3′ |
|---|---|
| sgRNA S. aureus | TCTGGAGCATTAGCGCTCGT (SEQ ID NO: 19) |
| 16S S. aureus | GGACGTATCATCGAACAAATCGGTACTTATAACCCAACGAGCGCT |
| | AATGCTCCAGAAATTAAAGTTGACGAAGCGTTAGCTTTAAAATGG′P |
| | (SEQ ID NO: 20) |
| S. aureus Forward | CATCTGCCTGAGTATGAAGGACGTATCATCGAACAAATCGGTA |
| | (SEQ ID NO: 21) |
| S. aureus Reverse | CTTGATGTCAGTTAGGACCATTTTAAAGCTAACGCTTCGTCAAC |
| | (SEQ ID NO: 22) |
| S. aureus Probe | TTCTGGAGCATTAGCGCTCGTTGGGTT (SEQ ID NO: 23) |
| sgRNA HIV | TAGCTACAGAGAAATCTCGA (SEQ ID NO: 24) |
| Proviral HIV | CACTTATCTGGGACGATCTGCGGAGCCTGTGCCTCTTCAGCTACCA |
| | CCGCTTGAGAGACTTACTCTTGATTGTAACGAGGATTGTGGAAC′P |
| | (SEQ ID NO: 25) |
| HIV Forward | CATCTGCCTGAGTATGAACACTTATCTGGGACGATCTGC (SEQ ID |
| | NO: 26) |
| HIV Reverse | CTTGATGTCAGTTAGGAGTTCCACAATCCTCGTTACAATC (SEQ ID |
| | NO: 27) |
| HIV Probe | TAGCTACAGAGAAATC (SEQ ID NO: 28) |

It is understood that the foregoing detailed description and accompanying examples are merely illustrative and are not to be taken as limitations upon the scope of the disclosure, which is defined solely by the appended claims and their equivalents. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A method of capturing circulating free DNA from a biological sample of a subject comprising:
forming a capture complex comprising an RNA-guided DNA binding protein, or a functional fragment thereof, bound to a guide RNA configured to at least partially hybridize to a circulating free DNA of interest;
incubating the capture complex with the biological sample to form a bound complex comprising the capture complex bound to the circulating free DNA of interest; and
removing remaining biological sample from the bound complex.

2. The method of claim 1, wherein the biological sample comprises blood or blood components.

3. The method of claim 1 or claim 2, wherein the blood component comprises plasma.

4. The method of any of claims 1-3, wherein the circulating free DNA is of microbial or viral origin.

5. The method of any of claims 1-3, wherein the circulating free DNA is circulating tumor DNA.

6. The method of any of claims 1-5, wherein the capture complex is linked to a solid surface.

7. The method of claim 6, wherein the solid surface is a particle.

8. The method of claim 7, wherein the particle is magnetic.

9. The method of any of claims 6-8, wherein the capture complex is linked to the solid surface by a biotin streptavidin linker.

10. The method of claim 9, wherein the RNA-guided DNA binding protein further comprises a biotin tag.

11. The method of any of claims 1-10, wherein the RNA-guided DNA binding protein comprises an epitope tag.

12. The method of any of claims 1-11, wherein the incubating comprises flowing the biological sample over immobilized capture complexes.

13. The method of claim 12, wherein the capture complexes are magnetically immobilized within or on the surface of a flow cell or a fluidic chamber.

14. The method of claim 12, wherein the capture complexes are immobilized through immunoprecipitation.

15. The method of any of claims 1-14, wherein the RNA-guided DNA binding protein is a CRISPR-associated (Cas) protein.

16. The method of any of claims 1-15, wherein the RNA-guided DNA binding protein is Cas9.

17. The method of claim 16, wherein the Cas9 is catalytically inactivated.

18. The method of any of claims 1-17, wherein the method further comprises:
purifying the circulating free DNA of interest;
amplifying the circulating free DNA of interest;
sequencing the circulating free DNA of interest; or
a combination thereof.

19. The method of any of claims 1-18, wherein the method further comprises analyzing at least a portion of the biological sample for the presence or absence of at least one biomarker.

20. The method of any of claims 1-19, wherein the method further comprises returning at least a portion of the biological sample to the subject.

21. A system for capturing circulating free DNA from a biological sample of a subject comprising:
an RNA-guided DNA binding protein, or a functional fragment thereof; and
a guide RNA configured to at least partially hybridize to a circulating free DNA of interest.

22. The system of claim 21, further comprising a flow cell or fluidic chamber.

23. The system of claim 21 or 22, further comprising at least one magnet.

24. The system of any of claims 21-23, further comprises a solid surface.

25. The system of claim 24, wherein the solid surface is a particle.

26. The system of claim 25, wherein the particle is magnetic.

27. The system of any of claims 21-26, wherein the RNA-guided DNA binding protein further comprises an epitope tag.

28. The system of any of claims 21-27, wherein the RNA-guided DNA binding protein further comprises a biotin tag.

29. The system of claim 28, wherein the RNA-guided DNA binding protein is linked to the solid surface.

30. The system of claim 29, wherein the RNA-guided DNA binding protein is tethered by a biotin streptavidin linker.

31. The system of any of claims 21-30, wherein the gRNA is bound to the RNA-guided DNA binding protein.

32. The system of any of claims 21-31, wherein the RNA-guided DNA binding protein is a CRISPR-associated (Cas) protein.

33. The system of any of claims 21-32, wherein the RNA-guided DNA binding protein is Cas9.

34. The system of claim 33, wherein the Cas9 is catalytically inactivated.

35. The system of any of claims 21-34, wherein the circulating free DNA is microbial DNA.

36. The system of any of claims 21-34, wherein the circulating free DNA is circulating tumor DNA.

37. The system of any of claims 21-36, further comprising the biological sample.

38. The system of any of claims 21-37, wherein the biological sample comprises blood or blood components.

39. The system of any of claims 21-38, wherein the blood component comprises plasma.

40. A device comprising at least one system as in claims 21-39.

41. The device of claim 40, comprising two or more systems, wherein each system comprises a gRNA configured to hybridize to a different circulating fee DNA of interest.

42. A kit comprising at least one system as in claims 21-39 or a device as in claims 40-41.

43. The kit of claim 42, comprising two or more systems, wherein each system comprises a gRNA configured to hybridize to a different circulating fee DNA of interest.


SEQUENCE LISTING (28 sequences)

SEQ ID NO: 1 - DNA, 20 bp, synthetic construct
  source 1..20: mol_type = other DNA
tagctacagagaaatctcga

SEQ ID NO: 2 - DNA, 90 bp, synthetic construct
  source 1..90: mol_type = other DNA
gacccactccatcgagatttctctgtagctagaccaaaatcacctatttttactgtgaggtcttcatgaagaaatatatctgaggtgtag

SEQ ID NO: 3 - DNA, 40 bp, synthetic construct
  source 1..40: mol_type = other DNA
catctgcctgagtatgaagacccactccatcgagatttct

SEQ ID NO: 4 - DNA, 46 bp, synthetic construct
  source 1..46: mol_type = other DNA
gttcagtgtagcttactgtatgaagacctcacagtaaaaataggtg

SEQ ID NO: 5 - DNA, 16 bp, synthetic construct
  source 1..16: mol_type = other DNA
tagctacagagaaatc

SEQ ID NO: 6 - DNA, 20 bp, synthetic construct
  source 1..20: mol_type = other DNA
tagctacagtgaaatctcga

SEQ ID NO: 7 - DNA, 86 bp, synthetic construct
  source 1..86: mol_type = other DNA
gtcccaggagaacgaagttgccgactccatcgagatttcactgtagctagaccaaaattattgtagagcagtgctgtcacaagcct

SEQ ID NO: 8 - DNA, 44 bp, synthetic construct
  source 1..44: mol_type = other DNA
gttgtcgatgctatctctaaatggatccagacaactgttcaaac

SEQ ID NO: 9 - DNA, 46 bp, synthetic construct; identical to SEQ ID NO: 4
  source 1..46: mol_type = other DNA

SEQ ID NO: 10 - DNA, 17 bp, synthetic construct
  source 1..17: mol_type = other DNA
ctagctacagtgaaatc

SEQ ID NO: 11 - DNA, 40 bp, synthetic construct
  source 1..40: mol_type = other DNA
catctgcctgagtatgaagtcccaggagaacgaagttgcc

SEQ ID NO: 12 - DNA, 45 bp, synthetic construct
  source 1..45: mol_type = other DNA
cttgatgtcagttaggaaggcttgtgacagcactgctctacaata

SEQ ID NO: 13 - DNA, 38 bp, synthetic construct
  source 1..38: mol_type = other DNA
catctgcctgagtatgaaagtctccactcacccaggaa

SEQ ID NO: 14 - DNA, 38 bp, synthetic construct
  source 1..38: mol_type = other DNA
gttgtcgatgctatctctagagctacgagctgcctgac

SEQ ID NO: 15 - DNA, 19 bp, synthetic construct
  source 1..19: mol_type = other DNA
ccctgaggcactcttccag

SEQ ID NO: 16 - DNA, 18 bp, synthetic construct
  source 1..18: mol_type = other DNA
catctgcctgagtatgaa

SEQ ID NO: 17 - DNA, 18 bp, synthetic construct
  source 1..18: mol_type = other DNA
gttgtcgatgctatctct

SEQ ID NO: 18 - DNA, 21 bp, synthetic construct
  source 1..21: mol_type = other DNA
gttcagtgtagcttactgtat

SEQ ID NO: 19 - DNA, 20 bp, synthetic construct
  source 1..20: mol_type = other DNA
tctggagcattagcgctcgt

SEQ ID NO: 20 - DNA, 90 bp, synthetic construct
  source 1..90: mol_type = other DNA
ggacgtatcatcgaacaaatcggtacttataacccaacgagcgctaatgctccagaaattaaagttgacgaagcgttagctttaaaatgg

SEQ ID NO: 21 - DNA, 43 bp, synthetic construct
  source 1..43: mol_type = other DNA
catctgcctgagtatgaaggacgtatcatcgaacaaatcggta

SEQ ID NO: 22 - DNA, 44 bp, synthetic construct
  source 1..44: mol_type = other DNA
cttgatgtcagttaggaccattttaaagctaacgcttcgtcaac

SEQ ID NO: 23 - DNA, 27 bp, synthetic construct
  source 1..27: mol_type = other DNA
ttctggagcattagcgctcgttgggtt

SEQ ID NO: 24 - DNA, 20 bp, synthetic construct
  source 1..20: mol_type = other DNA
tagctacagagaaatctcga

SEQ ID NO: 25 - DNA, 90 bp, synthetic construct
  source 1..90: mol_type = other DNA
cacttatctgggacgatctgcggagcctgtgcctcttcagctaccaccgcttgagagacttactcttgattgtaacgaggattgtggaac

SEQ ID NO: 26 - DNA, 39 bp, synthetic construct
  source 1..39: mol_type = other DNA
catctgcctgagtatgaacacttatctgggacgatctgc

SEQ ID NO: 27 - DNA, 40 bp, synthetic construct
  source 1..40: mol_type = other DNA
cttgatgtcagttaggagttccacaatcctcgttacaatc

SEQ ID NO: 28 - DNA, 16 bp, synthetic construct
  source 1..16: mol_type = other DNA
tagctacagagaaatc